(19) United States
(12) Patent Application Publication
(10) Pub. No.: US 2008/0317788 A1
(43) Pub. Date: Dec. 25, 2008

(54) Additive for Domestic Washing Processes

(76) Inventors: Rafael Louzan Garcia, Barcelona (ES); Simon Velarde Esquina, Barcelono (ES)

Correspondence Address: PATENT, COPYRIGHT & TRADEMARK LAW GROUP, 4199 Kinross Lakes Parkway, Suite 275, RICHFIELD, OH, 44286, US

(21) Appl. No.: 11/766,951

(22) Filed: Jun. 22, 2007

(51) Int. Cl. A61K 8/11; A61K 9/50; D06M 13/00

(52) U.S. Cl. 424/401; 424/402; 8/115.51; 8/115.6

(57) ABSTRACT

Additive for processes of domestic washing or others, and like cosmetics additive for shampoo and other compounds and of hygiene personal, susceptible to be incorporated to the process after the washing and to be clarified of the process that is constituted by a product micro-encapsulation chosen of between a cosmetic, a drug, or an evaporable product, being constituted the surrounding one of the microcapsules by a compound of different chemical nature. The additive is preferably in liquid form and of unitary doses, but it is possible also to obtain it in dust form. The invention also talks about to a procedure for the treatment of fibers (or skin or hair) with the additive.

### RELATED APPLICATIONS

The present invention is a Continuation in Part of U.S. Ser. No. 11/______, which was a National Phase filing of PCT/ES2005/012300 filed 16 Nov. 2005.

### BACKGROUND OF THE INVENTION

1. Field of the Invention

The present invention relates to an additive for domestic washing processes. It also relates to a process for treating fibres in a domestic washing process.

2. Description of the Related Art

In domestic washing processes, after washing with detergent to remove dirt from the clothing, the clothes are usually rinsed with products for special treatment, called products for the finishing treatment of washed clothes. For example, such as special treatment products usually used with fabric softeners which in turn incorporate some perfumed substance. However, in the case of perfumed softeners, this finishing cycle does not, on many occasions, have a long-lasting effect, with the washed clothes very quickly ceasing to emit the perfume they were impregnated with.

With the aim of prolonging the emission of perfume in fabric softeners, the perfume is contained in said softener in the form of microcapsules whose nucleus contains the desired perfume. An example of this type of fabric softener is disclosed in patent ES 2063241, wherein microcapsules and the fabric softeners containing them are described, to improve the finishing cycle of the clothing and prolonging their scent and feeling of freshness. Also, in WO 0162376, micro and nanocapsules are disclosed which are incorporated into detergents and cleaning products, for example, for the treatment of fabrics, such as mild detergents, stain removers, universal detergents, etc.

The microcapsules, or micrometric-sized capsules comprised of a nucleus covered in a coating suitable for holding the nucleus inside, are being used in many fields of the art. One of these is the finishing treatment of textiles or fibrous materials to give the fibres the required properties, for example, to make ironing easier, to make the fibres fire-resistant or water-proof, or to release a perfume or cosmetic agent of interest.

In this latter case, garments can be found on the market that incorporate a microencapsulated moisturizing and/or tonic agent, such as, for example, underwear with a microencapsulated firming agent which is released by the effect of rubbing against the wearer's skin and which comes into contact with the skin, being absorbed and, hence, releasing its effect. The use of microcapsules is done at manufacturing level, generally before the garments, that are to incorporate the microcapsules with the agent of interest, are made. An example of this is disclosed WO 03026594, wherein ginger oil is encapsulated in microcapsules which are applied to fabrics, more specifically in the manufacture of tights and/or socks, the ginger oil being released when it rubs against the skin, which enables improved microcirculation and reduces swelling of the lower extremities.

The problem with said garments is that once the microencapsulated active agent has been completely released, the garments or fibrous materials become conventional garments or fibres without the desired effects, because the microcapsules have opened and the reserves of the agent of interest used up, or because said agent has diffused through the barrier or coating of the microcapsules.

In an attempt to provide and prolong the emission of a determined perfume, U.S. Pat. No. 4,234,627 discloses a composition for the process of domestic pre-washing or washing which comprises a granular textile treatment mixture and an adjuvant for pre-washing or washing. The granular mixture contains some microcapsules and a so-called microcapsule transfer agent, which in practice refers to an organic material of the type conventionally used as domestic-use softening agents. However, in this case, the granular component with microcapsules is suitable for the pre-washing and washing process and subsequent drying of the clothes, incorporating a detergent or surfactant which is soluble in water. Said composition is sufficient for perfuming the garments that are washed, but it is a complex composition which has the added drawback that the user has to use the detergent or softener accompanying the granular mix.

The additive object of the present invention provides new solutions to said problems, while offering notable advantages to the state of the art.

### SUMMARY OF THE INVENTION

The additive for domestic washing processes, for incorporation in the process after washing and rinsing, is characterized in that it is comprised of a microencapsulated product which is either a cosmetic, a pharmacological drug, an evaporable product or a combination thereof, the microcapsule coating being comprised of a cationic compound.

According to another characteristic of the invention, the additive is in the form of a monodose.

The additive according to the invention is also characterized in that it is in liquid form.

According to another characteristic of the additive of the invention, the cosmetic product is selected independently from among a moisturizing compound, an ultra-violet ray protector, a toner, a product for reducing the effects of stress on the skin, an anti-wrinkle compound, a deodorant, a compound to slow hair-growth and/or a whitening compound.

The additive according to the invention comprises a pharmacological product which is selected independently from among a painkiller, a bactericide agent, an epulotic agent and/or essence of eucalyptus. According to another characteristic of the invention, the microcapsule coating is comprised of starch that has been subjected to a chemical modification to give it a positive charge, for example, via quaternization.

Another object of the invention is a process for treating fibres in a domestic washing process characterized in that after the stages of washing with detergent and rinsing of the fibres, one or more additives, separate from one another, are added, which are comprised of a microencapsulated product selected from among a cosmetic, a pharmacological drug, an evaporable product or a combination thereof, the microcapsule coating being comprised of a cationic compound.

It is envisioned that a combination of a structure of the walls of the microcapsules and the foregoing products of which the walls encapsulate provide a desirable combination of wall resistance during a preparation of the microcapsule and the treatment of the fibers during the domestic washing. It is anticipated that the wall comprises a capacity to reduce its resistance at the times subsequent to the washing. This reduction is resistance is accomplished by a variety of means, s.a., heating, humidity or, more commonly, friction of the fibers against tissue and/or the skin of the user. The wall of the microcapsule continues to weaken until it bursts to release the product therein. Because the microcapsules are impregnated within the fibers, the release of product provides a means to treat the user in contact with the fibers.

### DETAILED DESCRIPTION OF THE INVENTION

In general and as it has been described, there exist multitude of techniques and materials for the micro-encapsulation of functional compounds; they can possibly be applied (with additional advantages) to the washing additive purposes. In the same way, this additive can be obtained in different formats (liquid suspension/slurry or in powder). This way, it is possible to adjust the electrostatic properties of the surrounding material of the microcapsules, so that their sticky capacity to the textile material or others is variable, based on the anticipated final use. Thus, it is also possible to use the preparation of microcapsules directly in a powder form.

Next the materials of the capsule shell are described with greater details, and also the possible content of the capsule.

### The Materials of the Inside of the Microcapsules (Nucleus)

The products that can constitute the interior of the microcapsules are, evaporable cosmetics, pharmaceutical drugs or products that can evaporate; evaporable repellents products for insects or perfumes. As it has been described previously, most of the hydrophobic compounds can be encapsulated (a-polar), whereas they have not revealed to be reactive during the capsule manufacturing process; and also the practical totality of hydrophilic compounds (polar). For the first type, the perfumes are especially well-known invention, for their use in compositions for washing. This way, it is much more probable that the perfumes encapsulations stay active after the rinsing and drying procedures and therefore are apt to perfume clean and dry textile products, by transferring to these its odor properties.

A unique and specific advantage of the micro-encapsulation compounds is that the compounds which are reactive or volatile can be adhered (and be retained) to fibers, or in other organic materials such as hair or skin (in the case of cosmetic uses), so that these volatile compounds are not eliminated during the process of drying by centrifuged or contact with a hot and dry airflow. Those are defined as volatile materials that have a steam pressure superior to 3 μm of Hg at 25° C., and all the way up to 5000 μm. In the same way, it is possible to adhere and to retain compound with inferior steam pressures, by simple incorporation. Within those classified perfume compounds with average and high smell are certain that are used to improve the general sensation of freshness. In general, substantive perfumes are used, being those that contain a sufficient amount of ingredients that, used in normal levels in a product like a fabric softener, provide a smell that can be picked up by a person with normal smelling capacity.

This is a preferred aspect of the invention; it is not necessary to exclude certain types from perfumes that are not apt to be encapsulations by other known techniques; as for example, those perfume compounds that have a remarkable water solubility at certain temperature (ex. terpenic alcohols of low molecular weight, benzyl acetate or pheniletilic alcohol), or compounds with high boiling point like for example with temperature superior to 300° C. Therefore, the applied techniques allow the encapsulation of both lipo-soluble active principles and hydro-soluble active principles; and they also allow the use of microcapsules obtained in powder, suspension in liquids (slurry), as much as polar or a-polar capsules.

As it has been described previously, scent or pharmaceutical principles, cosmetic active ingredients can be encapsulated; or diverse chemical agents for the food sectors or the agricultural use. In each case, the combination of a structure of the walls of the microcapsules and the compound to micro-encapsulate, provide a desirable combination of wall resistance during the preparation of the microcapsule and the treatment of the tissue or the hair during the domestic washing or another one, and the capacity to reduce the resistance of the wall at later moments, by the use of a variety of means such as heating, humidity or, more commonly, friction of the fibers of tissue and/or the skin or the hair of the user of these textiles or another type of products.

If the material of the nucleus is not very volatile, additional more volatile materials can be added. Between those, they are hydrocarbons like octane, the decanis, the Dodecanese or the hex-Dodecanese, who increase the nature hydrophobic of the materials of the nucleus, and have very little scent. These compounds and others like fatlike acid, the limonene, saline and silicones and mixtures of them can be used in addition to modify the physical properties of the active principle to encapsulate (with respect to the thermal resistance, solidity to the washing, etc.) without affecting the functional effect of its own active principle. The objective is to have very strong walls during the process of elaboration and storage of the microcapsules, and then diminish the resistance of the wall at a precise moment and by the direct action of the media that varied, and to thus allow the nucleus to escape, slowly in the case of the invention object of this description, the active principle contained in the capsules.

Optional “helping like” ingredients can be added like: viscosity agent, anti-oxidant, bactericidal, fungicide, moisturizer, conservatives, polishers, opalinificadores, anti-freeze, anti-shrinking agents and active ingredients that provide ironing facility; always based on the anticipated final use for the microcapsules.

### The Material Composition of the Microcapsule Wall

The materials used to form the wall are diverse, depending on the method selected for the preparation of the microcapsules and the active principle that is desired to be encapsulated. These materials have been described already in numerous previous patents. The families of materials used for the preparation of the envelopes of the microcapsules can be grouped of the following form:

### 1.—Melamine and Derivatives

They are melamine based, and its derivatives like: polyurethanes, polyamides and polyesters. Usually, the most used ones are the polymeric amino plastics that include reactive products like formaldehyde. These materials can be polymerized into acid conditions from a previous stage of soluble polymerization in water (U.S. Pat. No. 4,234,627). These pre-polymers are prepared with a formaldehyde reaction and urea in mole razones 1:1,2 to 1:2,6. As modifiers of the urea can be added tiourea, Ciano amide, guanidine, N-alquil urea, phenol, sulfonamides, anilines and other amines.

In general, the melamine-formaldehyde mixtures present/display the advantage of a greater resistance to the chemical or thermal degradation; and a greater uniformity in the size of obtained particles. On the contrary, its capacity of electrostatic adhesion to textile fibers or hair is lower, and must properly be employed.

It has been observed that these microcapsules are trapped between fibers of the washed fabric during the process of domestic washing, and later an action of shear or crushing is required to break the wall of the microcapsule and to therefore release the contained product in its wall. In this case, the duration of the aroma effect is more persistent throughout the time since all the microcapsules are not broken simultaneously, so that it sustains a minimum of about 6-10 washings.

### 2.—Carbohydrates of Cationic Starch.

A material of extensive use is polysaccharide like the starch, or any carbon hydrate type starch (potato or tapioca, between the most common), which must be set with positive charge (cationization) to be able to be stick to fibers of the fabric or the skin and hair, that are generally charged negatively. For example, in the use of starch, it can be set with positive charges through a treatment of quaternization with sulfate of dimetil.

### 3.—Gelatin and Vegetal Derivatives

The envelop employed can be gelatin coercer with a poly-anion like gum arabige and a reticulate agent like the gluto-aldehide one (or alternatives of vegetal origin like the carragenatos); also apt being composed like poly-phosphate, alginate, cellulous carragenine, poly-acrylic, carboximetile, pectin, and their mixtures.

This type of microcapsules has the particularity, unlike all the previous cases, that it is not constituted by one surrounded capsule and a nucleus space of different chemical nature in which the desired active principle is contained; instead the whole microcapsule is formed by a gelatin matrix, within which they are included the molecules of the desired active principle. Therefore, the liberation of the active principle does not take place by breakage of the microcapsule (all the active principle of a single microcapsule is freed simultaneously) but instead that is taken place as the microcapsule is degraded, and the molecules of active principle are being exposed to the outside. This way the effect of the active principle is perceived more slightly, but during a longer time.

The size of the microcapsules thus obtained is generally much greater. Plus, in this case, the microcapsules can protect the active principal of the chemically aggressive surroundings (like for example the interior of the stomach or intestinal tract, in medicinal uses or for pro-biotic foods), and the liberation of the active principle can be controlled (when the degradation of the microcapsule begins) once the surroundings have degraded.

Also, the use of materials from animal origin like the gelatin can be contraindicated in specific cases and/or markets. In this case, it is possible to use contratipos of vegetal use, like the carragenatine and alginate aneriormente mentioned; that they have a very similar chemical nature.

### 4.—Silicones

Compounds of the family of silicones are also used; the silicone resins and the resins epoxy (U.S. Pat. No. 3,870,542 Going ET al., 1975). In this case, the advantage is had that the microcapsules can be used with all types of reactive groups, so that it is also possible to adjust the level of adhesion and the shear strength with much precision.

5.—Organic Compounds with the Ability to Form Bi-Layers.

Organic compounds can possibly be used; the corresponding molecules have their two ends with a different ionic character (for example the fatty acids), having one part hydrophobic (a-polar) and another one hydrophilic (polar); so that in the process of synthesis of the membrane, a bi-layer is obtained between the external surface in contact with the active ingredient and the internal surface of the capsule (the light of the interior of the microcapsule), having both the same ionic character, has another space (of few microns of thickness) of noticeable different character. In this case, the active ingredient contained inside the microcapsule is protected by a physical barrier (the wall of the microcapsule) and simultaneously an electrical barrier (the bi-layer of the wall), which supposes interesting possibilities of application for the controlled liberation of the active ingredient.

Using all the combinations, it is possible to contain the isolated the active ingredient of the media; be the polar or a-polar active principle, and the media are polar or a-polar (that is to say, an a-polar active ingredient isolated of an media a-polar or polar; and a polar active ingredient isolated of an media a-polar or polar).

This type of microcapsules presents the advantage that allows adjusting the thickness of the bi-layer (i.e. from the length of the end hydrobo of the fatty acid molecule) and its size and resistance; and it is possible to produce capsules very small.

On the contrary, they are very sensible to the changes of ionic force in the media, which can be used to fit a specific speed of degradation/decay. In this case, the degradation/decay processes is chemically based; the physical effect of the produced forces of shears in the friction is not as important here.

### The Liquid Vehicle

In general, the presentation of these compounds for its use as additive in the end item, is as a liquid product, in which the microcapsules are in microscopic suspension in a solvent which he is compatible with the liquid in which it is due to mix.

In addition, of preferred way the additive is provided in a suspension form of the microcapsules in an inert liquid, as water. The water can be distilled, de-anodized. In necessary case and based on the nature of the active ingredient and the properties of the microcapsules compounds equipped with acidity to protect the polar envelop of the microcapsules are added. In such case, a habitual composition is the one that includes between 0.1% and a 40% in weight of the active ingredient microencapsulated; between 1.0% and 20% in weight of the membrane; and a sufficient amount for the 100% in weight of water, that can include other dissolved compounds.

Also they are useful as liquid vehicle mixtures of water and up to 20% of alcohol or poly-alcohols (poliole) of short chain like isopropanol, ethanol, etilen-glycol, butanol, propilenglycol, dietilenglycol and similar, and their mixtures.

Finally, also the suspension of the microcapsules in polar liquids like an organic dissolvent is possible, any of them can be used, and the most common being alcohol and glycerin.

### The Solid Vehicle

The additive is also provided in solid form, in which the microcapsules appear in powder format, of different particle size. The solid format is obtained by means of a process of drying, following the obtaining of the suspension of microcapsules. The conditions of the process depend on the type of envelop used, and is function of the conditions of the process of the microcapsules and the conditions of the drying, it is possible to fit different particle size to obtain powder.

This way, the used techniques as they are described in the present invention directly allow the use of the microcapsules like powder, with which the rank of properties, benefits and applications increases considerably. Thus it is possible, for example, to increase the effectiveness of the treatment in the case that requires the use of a type of microcapsule that does not emphasize by its capacity of adhesion to textile fibers or hair; since it is possible to load much more active ingredient in a dose for a final use like suspension in spray or to prepare a liquid suspension highly concentrated, and to assure therefore the effect desired during sufficient time.

### The Preparation Method for the Microcapsules

The microcapsules are obtained on the basis of different preparation methods that already have been properly described in patents and scientific Literature. These methods of fabrication are grouped in two categories, the chemical methods by separation between phases, and the physical methods.

For the first method, there are a colloidal accumulation (coacervation), the interficible polymerization, and the formation of liposome or bi-layer. The presence of a reticulante or polymerizing agent is required and a temperature of reaction between 30 and 80° C. The time of preparation oscillates between the 30 minutes and the 24 hours.

For the second method, there is a co-extrusion (in concentric jets), the use of fluidizised beds, and by means of the use of revolving disc.

Next are described the basic characteristics of the microcapsule production and the general methods of preparation.

### Melanin and Derivatives

The interficible polymerization process (polymerization in situ) is described with greater detail in U.S. Pat. No. 3,516,941 (Matson, 1970); in that a pre-condensed urea-formaldehyde solution (metilol urea) is formed containing 3 to 30% of pre-condensed (interficible polymerization of urea/formaldehyde). The insoluble active ingredient disperses through this solution and forms discreet micro-drops. While maintaining the temperature of the mixture between 30 and 80° C., acid is added to obtain the polymerization of the dissolved pre-condensed. Shaking vigorously, the polymerization takes place at the same time that the insoluble active ingredient is retained inside the formed microcapsules.

The water insoluble active ingredient can directly be added to the media, or through particles that works like transfer agents to the capsule. In general, the water insoluble materials (less of 5% in weight at 30° C.) have a point of fusion between 40 and 150° C. In these, there is an imidazolinio, a production of quaternary ammonium salts with fatty alquil radicals, or alquil salts piridinio (U.S. Pat. No. 3,686,025, Morton, 1972 and U.S. Pat. No. 4,269,727, U.S. Pat. No. 4,303,548, and U.S. Pat. No. 4,460,722).

As coacervantes or reticulantes agents one can use, among others, resins of formal-urea, glutaraldehyde, poly-glycols or others.

The average size of the obtained microcapsules by this mean are from 3 to 50 μm, the thickness of the wall is from 0.1-2 μm, and in general can contain in their interior an amount of active principle around the 35-90% in weight.

### Carbohydrates of Cationic Starch

These microcapsules are prepared by coacervation of carbohydrate that previously has undergone a cationization process, by means of the reaction with a strongly positively loaded molecule (for example any type of quaternary ammonium or its derivatives).

The average size of the obtained microcapsules by this mean are from 10 to 50 μm, the thickness of the wall is from 0.2-1.5 μm, and in general can contain in their interior an amount of active principle around the 4-18% in weight.

### Gelatin and Vegetal Derivatives

These microcapsules are prepared by coacervation (complex) of the gelatin with arabiga gum. The arabiga wall of gelatin/gum interacts with the fabric softener matrix, so that there is an interchange of ionic species with the electrolyte or the tension-active of fabric softener or detergent products of the formula. A swelling of the wall of the microcapsule takes place; then that softens it without breaking it, which allows it to attach itself between fibers of the fabric during the cycles of rinsing. In the later process of the drying and by effect of the heat, the contained active ingredient inside the microcapsules tends to expand and finishes breaking the microcapsule. This effect takes place gradually, so that—unlike previous innovations, the scent effect of the perfume stays for more of one washing process.

The average size of the obtained microcapsules by this mean are from 200 to 500 μm; the thickness of the wall can not be discussed because a uniform matrix is used, and in general can contain in their interior an amount of active principle around the 30-50% in weight.

### Silicones

These microcapsules are prepared by interficible polymerization. The work method is very similar to the one used for melamine, although in this case it is possible to polymerized the framework of silicone to a quite inferior temperature, between 20 and 40° C. This presents the advantage that, unlike what happens with the microcapsules of melamine, there is less risk of damaging those molecules of the active ingredient that are especially sensible to the temperature.

In addition, they do not contain residue of formaldehyde that—unlike the melamine microcapsules—could make them apt for cosmetic and pharmaceutical uses in which the presence of formaldehyde residual is not allowed.

The average size of the obtained microcapsules by this mean are from 4 to 10 μm, the thickness of the wall is from 0.1-0.8 μm, and in general can contain in their interior an amount of active principle around 35% in weight.

Organic Compounds with the Ability to Form Bi-Layers.

These microcapsules are produced through a somewhat different coacervation process, since in this case, the natural properties of hydrophobic and hydrophilic of the compound that forms the membrane of the microcapsule (for example a fatty acid); it allows the production of microcapsules in unique reliable solvent, to which the active principle to encapsulate is added (it must have a polar nature different from media) and the component of the membrane, is to say fatty acid.

This process can be done at room temperature and the same material of the membrane can serve to encapsulate hydrophobic and hydrophilic active ingredients. On the contrary, its resistance to the physical impacts and tensions of shears are very poor, and in addition they are very sensible to the changes of polarity of the media that contain them.

However, this same fact can be used for cosmetic and pharmaceutical, in which the precise liberation of the active ingredient needs in media of certain polarity (habitually inside a biological organism) after having crossed other means in which the active ingredient had been chemically degraded,

Its capacity of adhesion to fibers and hair is not too elevated, and its capacity to be modified chemically to improve this point is not much weighed either; that forces these processes to be developed before producing the microcapsules.

The average size of the obtained microcapsules by this mean are from 0.5 to 10 μm, the thickness of the wall is from 0.1-0.3 μm, and in general can contain in their interior an amount of active principle around the 40-95% in weight.

### EXAMPLES

### Example 1

### Additive with Cosmetic Product with Moisturizing Effect

60% -85% in weight of water on the additive gross weight.

The microcapsules constituted by a surrounding of melamine in a percentage in weight, respect the total of the additive, 5% to 10% and an active ingredient from 10% to 30% in weight of extract of Aloe Vera (aloina, C₂₁H₂₂O₉, 1,8-dihidroxi-10-(β-D-glucopiranosil)-3-(hidroximetil)-9(10H) antraquinone; CAS No. 1415-73-2) y chitosan ([C₆O₃H₁₀N]ₙ, poly[β-(1-4)-2-amino-2-deoxi-D-glucopiranosa]), with respect to the total of additive (FIG. 1).

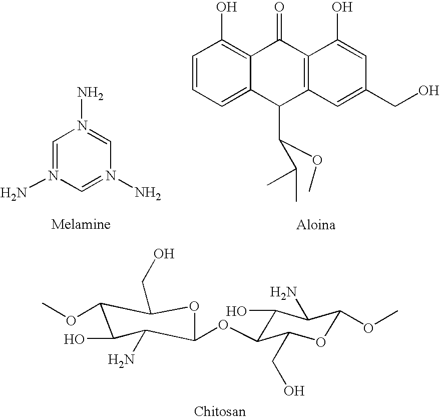

### Example 2

### Additive with Cosmetic Product with Photo-Protector Effect

60% -85% in weight of water acidic on the additive gross weight.

The microcapsules constituted by a surrounding of quaternized starch in a percentage in weight, respect to the total of the additive, 10% to 20%; and an active ingredient from 5% to 15% in weight of extract of Aloe Vera (aloína, C₂₁H₂₂O₉, 1,8-dihidroxi-10-(β-D-glucopiranosil)-3-(hydroximetil)-9(10H) antraquinona; CAS No. 1415-73-2), chitosan ([C₆O₃H₁₀N]ₙ, poly[β-(1-4)-2-amino-2-deoxi-D-glucopiranosa]) y Cinamato (cinamato de metilo, C₁₁H₁₂), with respect to the total of additive (FIG. 2).

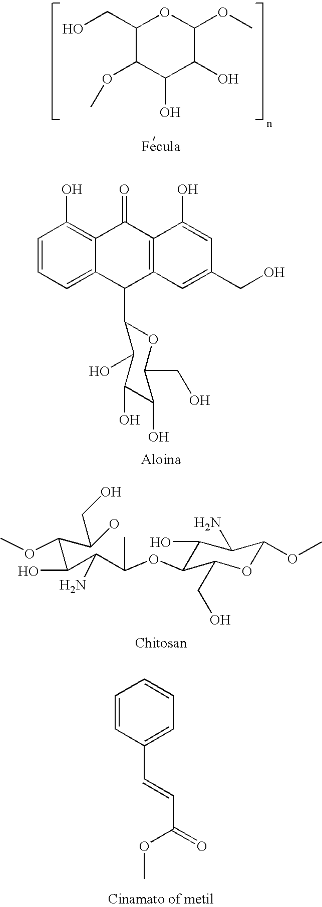

### Example 3

### Additive with Cosmetic Product with Anti-Cellulite Effect

70% -94% in weight of water on the additive gross weight.

The microcapsules constituted by a surrounding of quaternized starch in a percentage in weight, respect to the total of the additive, 5% to 20%; and an active ingredient with proliposomas, carnitine (C₇H₁₅NO₃), escine (C₅₅H₈₆O₂₄, 2-metil-(3-β, 4-β, 16-α, 21-β, 22-α)-22-acetoxi-3-[[O-β-glucopiranosil]-(1-2)-O[β-D-glucopiranosil-(1-4)]-β-D-glucopiranurosil[oxi]-16,23,28-trihidroxioleano-12-en-21-il-ester(2E)] of acid 2 benzoic), hydroxide de tetraetilamonio and/or caffeine (C₈O₂N₄H₁₈), with respect to the total of additive (FIG. 3).

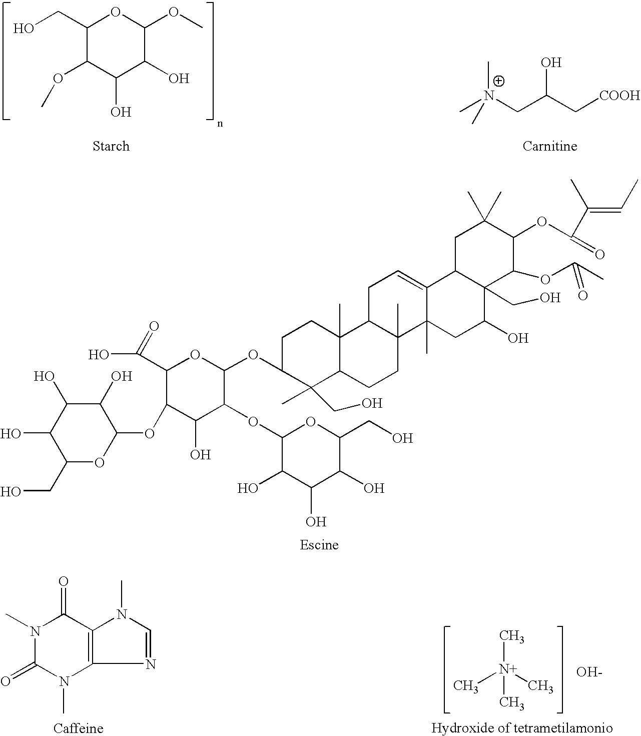

### Example 4

### Additive with Cosmetic Revitalizing Anti-Wrinkles for the Skin

80% -98% in weight of acidic water on the additive gross weight.

The microcapsules constituted by a surrounding of quaternized starch in a percentage in weight, respect to the total of the additive, 1% to 5%; and an active ingredient of extract of Aloe Vera (aloína, C₂₁H₂₂O₉, 1,8-dihidroxi-10-(β-D-glucopiranosil)-3-(hidroximetil)-9(10H) antraquinona; CAS No. 1415-73-2), and/or ubiquinona (C₂₄O₄H₃₁) and/or retinol (C₂₀H₃₀O, CAS No. 11103-57-4) and/or astaxantina (3,3′-dihidroxi-4,4′-diceto-β-caroteno; C₄₀H₅₁O₄) in a percentage in weight, on the gross weight of the additive, 1% to 5% (FIG. 4).

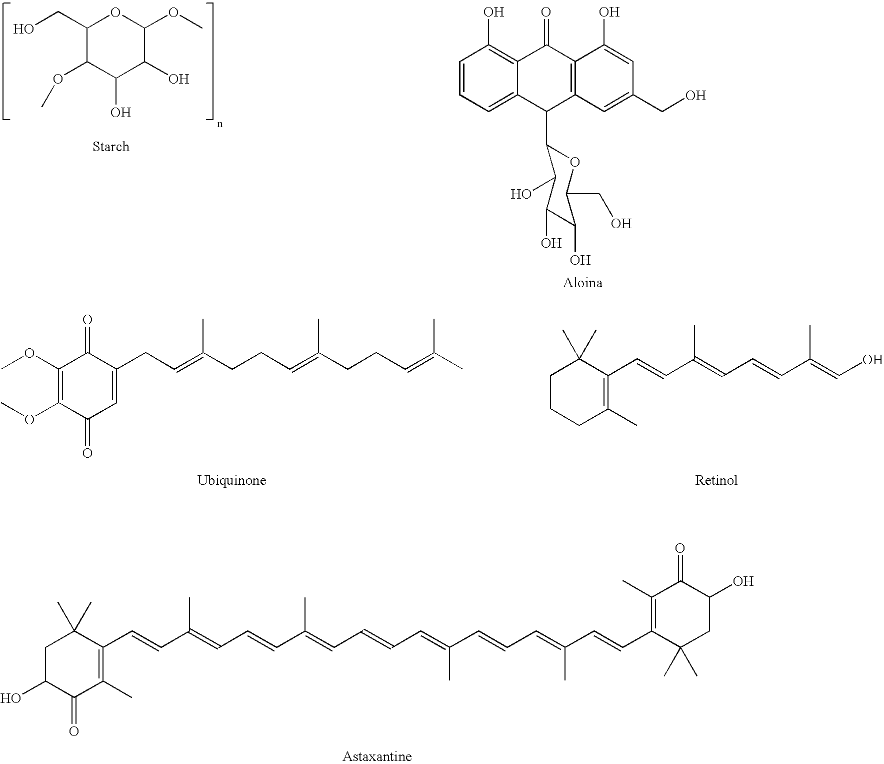

### Example 5

### Additive with Cosmetic Anti-Stress

80% -98% in weight of water with respect to the additive gross weight.

Microcapsules constituted by a surrounding one of silicone in a percentage in weight from 5% to 25%; and by an active principle that represents between 10% and 35% in weight on the total of additive and that includes extract of Kava (Piper methysticum) and/or serotonin (5-hidroxi-triptamina, C₁₀ON₂H₁₂). (FIG. 5).

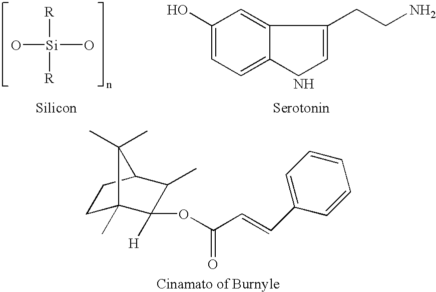

### Example 6

### Additive with Essence of Eucalyptus

40% -85% in weight of water, with respect to the gross weight of the additive for domestic processes of washing.

Microcapsules with a surrounding one of silicone, in a percentage in weight respect the total of additive from 5% to 20%; and with an active principle from 10% to 40% in weight on the total of additive, extract of eucalyptus (FIG. 6).

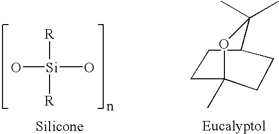

### Example 7

### Additive with Energy Properties

80% -98% in weight of water (on the additive gross weight).

Microcapsules with surrounding of gelatin in a percentage in weight with respect to the total of additive, between 1% and 15%; and active principle that includes essence of ginseng and moisturizer (extract of Aloe side, aloine, C21H2209, 1,8-dihidroxi-10-(βD-glucopiranosil)-3(hidroximetil)-9(10H) antraquinone; CAS Not 1415-73-2), in a percentage in weight with respect to the total of additive, 1% to 5%. (FIG. 7).

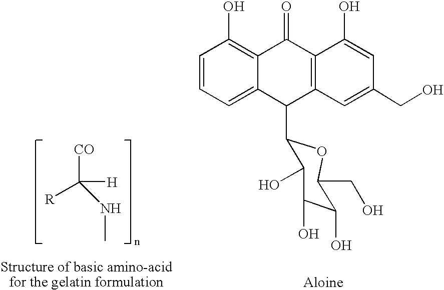

### Example 7

### Additive with Insect Repellant

70% -94% in weight of water, with respect to the gross weight of the additive.

Microcapsules with surrounding of melanin that contains a mosquito repellant (tolu-amide) from 1% to 10% of the gross weight of the additive, and with a surrounding from 5% to 20% in weight (FIG. 8).

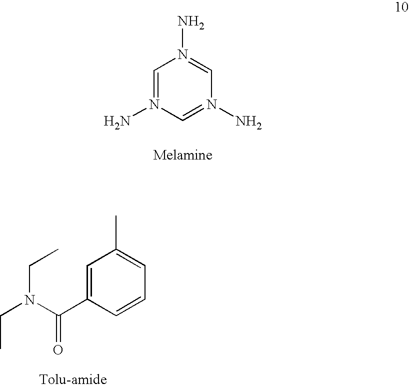

### Example 9

### Additive with Cosmetic Product having Anti-Cellulite Effect

94% -100% in dry weight of the gross weight of the additive.

Microcapsules constituted by a bi-layer surrounding (membrane) lipidic (on the basis of fosfoetidil-etanolamine) in a percentage in weight, respect to the total of the additive, 25% to 30%; and an active principle with proliposomes, camitine (C₇H₁₅NO₃), escine (C₅₅H₈₆O₂₄, 2-metil-(3-β, 4-β, 16-α, 21-β, 22-α)-22-acetoxi-3-[[O-β-glucopiranosil]-(1-2)-O[β-D-glucopiranosil-(1-4)]-β-D-glucopiranurosil[oxi]-16, 23, 28-trihidroxioleano-12-en-21-il-ester(2E)] of benzoic acid 2), hydroxide of tetra-etil-amonio and/or caffeine (C₈O₂N₄H₁₈), from 75% to 70% in weight on the additive gross weight (FIG. 9).

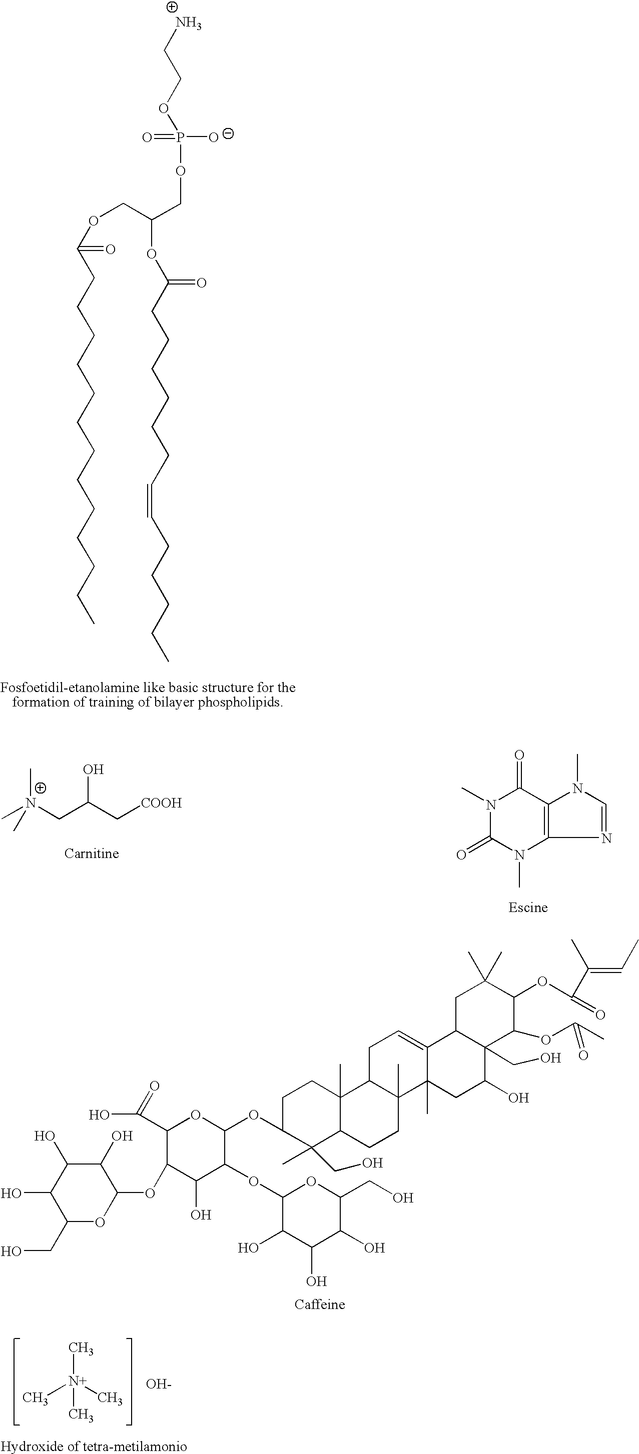

### Example 10

### Additive with Revitalization Cosmetic Anti-Wrinkles of the Skin

88% -98% in dry weight, respect the gross weight to the additive.

Microcapsules of silicone with surrounding that constitutes between 56% and a 15% in weight on the total of additive; and active principle that includes extract of Aloe Vera (aloine, C₂₁H₂₂O₉, 1,8-dihidroxi-10-(β-D-glucopiranosil)-3-(hidroximetil)-9(10H)antraquinona; CAS No. 1415-73-2), and/or ubiquinone (C₂₄O₄H₃₁) and/or retinol (C₂₀H₃₀O, CAS No. 11103-57-4) and/or astaxantine (3,3′-dihidroxi-4,4′-diceto-β-caroteno; C₄₀H₅₁O₄), in a percentage in weight, on the gross weight of the additive, 44% to 85% (FIG. 10).

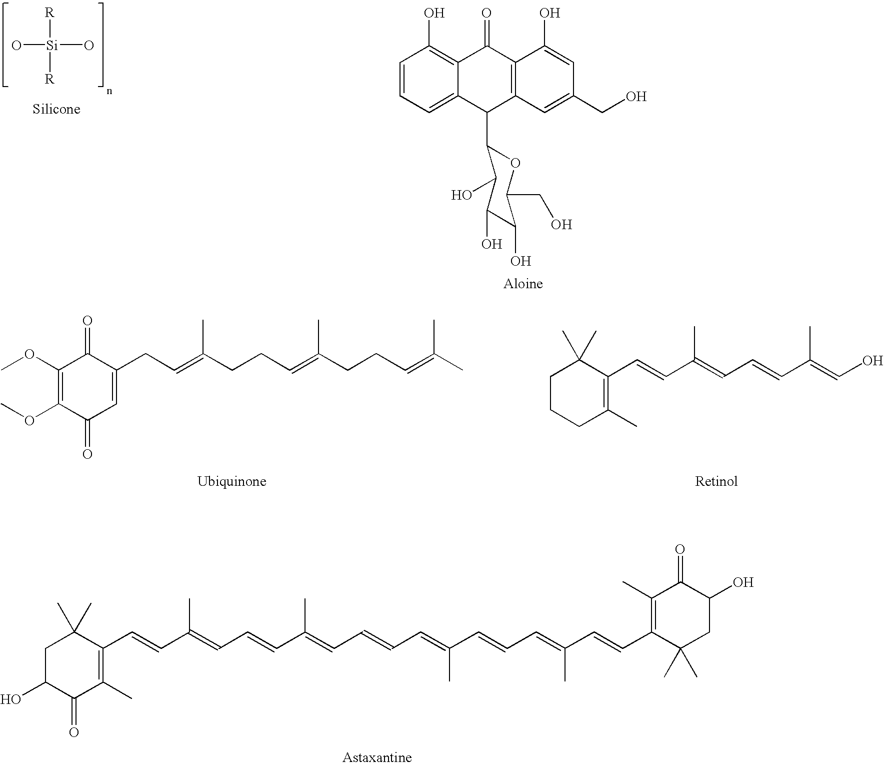

### Example 11

### Additive with Cosmetic Anti-Stress

80% -98% in dry weight.

Microcapsules constituted by a surrounding of melamine in a percentage in weight from 16% to 25%; and by an active principle that represents between 75% and 86% in weight on the total of additive and that includes/understands extract of Kava (Piper methysticum) and/or serotonin (5-hidroxitriptamina, C₁₀N₂H₁₂). (FIG. 11).

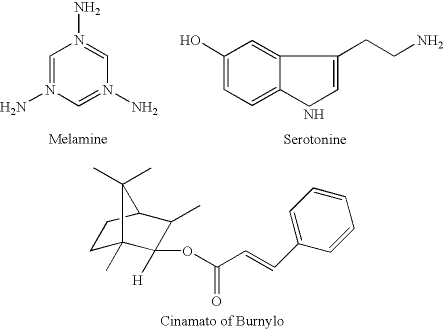

In all the examples, the cationic microcapsule coating corresponds to any type of quaternized starch, of the type known by persons skilled in the art, e.g. leguminous plants, cereal starch, etc., such as maize, rice, tapioca, which are water-soluble. Fatty acids that have been modified to give them positive charges can also be used.

The additives in the examples 1 to 10 are usually, for example, for use with underwear, socks, tights, and even sheets. All the cosmetic, pharmacological and evaporable products are used in the form of microcapsules with the fibers of garments that, once washed and rinsed, are subjected to the action of the additive according to the invention. Once dry, the garments retain the microcapsules, with the products of interest inside them, so that, by the effect of rubbing against the skin or movement of the user, the microcapsules break and the product inside is released.

The additive object of the invention is intended for insertion in the compartment used for additives for treating clothes, once washed and rinsed, where fabric softeners are generally added. The advantage of this additive is that the user can use it separately from the fabric softener to give the clothing the desired finish. Additives according to Examples 1 to 5 can thus be used when one is seeking a continuous, long-lasting application of a certain cosmetic with the clothing that is used by the user, e.g. a moisturizer for the legs which is released while the user is wearing tights which, beforehand, were treated for this purpose.

In other cases, the user can treat sheets so that a particular fragrance or essence is evaporated on a continuous basis. In addition, during the domestic treatment process, various additives can be applied to the garments at the same time, for example, an additive with microcapsules with a moisturizing product and an additive with microcapsules with a photo-protective product. For this, the formulation of the additive in a single-dose is sufficient for the user to make up the desired combination as required. The additive object of the invention is designed so that it adheres between clothing fibres at the working temperatures and speeds of domestic washing machines. Additives with microcapsules can thus be used up to 60° C. without compromising their consistency and, at the same time, favoring their impregnation into the fibers. Once the additive has been applied, the garment can be dried or ironed up to a temperature range of between 140° C. and 150° C. Once the user has worn the garment treated with the additive object of the invention, it is recommended not to wash it at more than 30° C. to prolong the effect, which does not generally run out until after 4 or 5 washes.

Obviously, the additive can also be applied in cases where hand-washing of garments is preferred. In this latter case, in order to ensure that the microcapsules are incorporated uniformly and to achieve high performance (impregnation of the fibres with a higher number of microcapsules), it is recommended to leave the garment treated in the additive for at least 10 to 15 minutes, stirring from time to time. The additive for domestic washing processes, in addition to using an additive with a formulation that is not complex, leads to positive results deriving not solely from the cosmetic or pharmacological effect it has, but also in terms of its long-lasting effect. In this way, it should be highlighted that, with a treatment of this type, the garments manage to exert the desired effect, whether they are moisturizing or photo-protective products, an insecticide, painkiller, etc., at least for four washes without subsequent treatment with additives. The additive for domestic washing processes, object of the invention, may be added in the form of a fine powder, the microcapsules having a granular size of between 5 μm and 15 μm, which is also the size of the microcapsules when they are applied in suspension in a liquid vehicle. Once the clothing has been washed and rinsed, the same water that enters the washing-machine compartment used to put all additives for special treatment of garments, is responsible for incorporating the powder particles or microcapsules therein, making up the liquid medium that will come into contact with the fibers. This allows for a more uniform distribution of the additive in all the clothes.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents. Therefore, the scope of the invention is to be limited only by the following claims.

Having thus described the invention what is claimed as new and desired to be secured by Letters Patent is as follows:

1. Additive for domestic washing processes, for incorporation in the process after washing and rinsing, characterized in that it is comprised of a microencapsulated product which encapsulates a cosmetic, a pharmacological drug, an evaporable product or a combination thereof, the microcapsule coating being comprised of a cationic compound.

2. Additive according to claim 1, characterized in that it is in the form of a single-dose.

3. Additive according to claims 1, characterized in that it is in a form selected from the group comprising: solids; and liquids.

4. Additive according to claims 1, characterized in that the cosmetic product is taken separately from among a moisturizing compound, an ultra-violet ray protector, a toner, a product for reducing the effects of stress on the skin, an anti-wrinkle compound, a deodorant, a compound to slow hair-growth and/or a whitening compound.

5. Additive according to claims 1, characterized in that the pharmacological product is selected separately from among a painkiller, a bactericide agent, an epulotic agent and/or essence of eucalyptus.

6. Additive according to claims 1, characterized in that the microcapsule coating is comprised of quaternized starches.

7. Process for treating fibres in a domestic washing process, characterized in that after the stages of washing with detergent and rinsing of the fibres, one or more additives, separate from one another, are added, which are comprised of a microencapsulated product selected from a cosmetic, a pharmacological drug, an evaporable product or a combination thereof, the microcapsule coating being comprised of a cationic compound.

8. Additive according to claim 2, characterized in that it is in the form of a liquid.

9. Additive according to claim 2, characterized in that the cosmetic product is taken separately from among a moisturizing compound, an ultra-violet ray protector, a toner, a product for reducing the effects of stress on the skin, an anti-wrinkle compound, a deodorant, a compound to slow hair-growth and/or a whitening compound.

10. Additive according to claim 3, characterized in that the cosmetic product is taken separately from among a moisturizing compound, an ultra-violet ray protector, a toner, a product for reducing the effects of stress on the skin, an anti-wrinkle compound, a deodorant, a compound to slow hair-growth and/or a whitening compound.

11. Additive according to claim 2, characterized in that the pharmacological product is selected separately from among a painkiller, a bactericide agent, an epulotic agent and/or essence of eucalyptus.

12. Additive according to claim 3, characterized in that the pharmacological product is selected separately from among a painkiller, a bactericide agent, an epulotic agent and/or essence of eucalyptus.

13. Additive according to claims 2, characterized in that the microcapsule coating is comprised of quaternized starches.

14. Additive according to claims 3, characterized in that the microcapsule coating is comprised of quaternized starches.

15. An additive in the form of an insoluble microcapsule that is impregnated within fibres during a wash cycle, said microcapsule comprises:
a cationic coating; and,
an evaporable product encapsulated within said cationic coating;
wherein a structure of the walls of said microcapsule and said product provide a desirable combination of wall resistance such that said walls reduce said resistance at times subsequent to washing, said wall weakens until said microcapsule bursts to release said product therein to provide a means to treat a user in contact with said fibres.

16. The additive of claim 15, wherein said resistence is weakened by means of a heat, a humidity or a friction of said fibres against said user.

17. The additive of claim 15, wherein said evaporable product is selected from a group comprising:
an insect repellant;
a perfume;
an anti-wrinkle cream;
a cosmetic;
a whitener; and,
a pharmaceutical.

18. The additive of claim 15, wherein said cationic coating is a quarternized starch.

19. The additive of claim 18, wherein a quarternization with dimethyl sulphate provides a means to manufacture said cationic coating.

20. The additive of claim 15, wherein said microcapsules are comprised in the following forms.
suspended in an inert liquid;
a fine powder obtained by a process of drying.

21. The additive of claim 15, wherein said microcapsule approximates a granular size between 5 μm and 15 μm.
